(19) United States
(12) Patent Application Publication
(10) Pub. No.: US 2006/0198877 A1
(43) Pub. Date: Sep. 7, 2006

(54) Medical instrument for autologous chondrocyte transplantation

(75) Inventors: Matthias Steinwachs, Freiburg (DE); Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(73) Assignee: Karl Storz Gmbh & Co. KG Germany

(21) Appl. No.: 11/367,678

(22) Filed: Mar. 3, 2006

(30) Foreign Application Priority Data
Mar. 3, 2005 (DE) 10 2005 010 988.8

(51) Int. Cl. A61B 17/50; A61K 9/70

(52) U.S. Cl. 424/443; 606/131

(57) ABSTRACT

A medical instrument for autologous chondrocyte transplantation comprises a stamp for introducing an implantable nonwoven into a cartilage. This stamp has, on a distal end face, at least one opening to which an underpressure can be applied.

### CROSS-REFERENCE TO RELATED APPLICATIONS

The present application claims priority of German patent application No. 10 2005 010 988.8 filed on Mar. 3, 2005.

### BACKGROUND OF THE INVENTION

The invention relates to an instrument for autologous chondrocyte transplantation.

Autologous chondrocyte transplantation (ACT) is a method for the repair of cartilage damage in the human body.

The articular cartilage in the area of the human knee differs in thickness according to the topography. In the area of the patella, it can reach a layer thickness of 7 to 8 mm. Since the articular cartilage does not have any direct vessel or nerve attachments, it is nourished mainly through diffusion from the synovial fluid of the intraarticular space. The cross-linking of various matrix components to form the cartilage ground substance permits mechanical damping and almost frictionless sliding of the articular surfaces. At the cellular level, there is a complex structure of cartilage cells (chondrocytes), collagen fibers and proteoglycans. The healthy cartilage in the area of the knee of a human adult is able to tolerate loads that can amount to a multiple of the body's weight.

Damage to the articular cartilage represents a major problem in routine traumatology and orthopedics. The limited healing capacity of cartilage has long been known and is due essentially to the latter's particular structure and anatomy.

Damage to the articular surface, above all in the area of the load-bearing zones of the sliding surface of the joint, therefore entails increased risk of substantial joint damage in the sense of premature arthrosis. Known methods for biological reconstruction of full-layer cartilage damage are in most cases suitable only for small to medium-sized defects. In full-layer cartilage damage, above all in the area of the knee joint, with a defect area covering more than ca. 4 cm², there has therefore been increasing clinical use of autologous chondrocyte transplantation.

In this method, cartilage tissue is removed, under arthroscopy, from joint parts that are not load-bearing. Cartilage cells are isolated from this tissue and cultured in a cell culture.

This culturing of the cartilage cells is increasingly being done within a nonwoven-like implant, for example a collagen matrix. Such a nonwoven provides the cartilage cells with a three-dimensional structure on which they are able to grow and spread.

A nonwoven obtained in this way and occupied by chondrocytes is then transplanted into the cartilage defect zone during a second intervention.

During this second intervention, the margins of the cartilage defect are dissected, i.e. the margins of the defect area are cut out and the bone in the area of the defect is exposed. In this connection, particular care has to be taken to ensure that the margins of the defect area are cleanly dissected in order to permit smooth attachment of the implant.

A template of the defect is now usually prepared from a sterile material, and the implantable nonwoven is cut to size with the aid of this template. This implantable nonwoven cut to size in this way is then fitted into the cartilage defect.

This procedure is generally performed by arthrotomy, that is to say by means of a semi-open procedure. Such a semi-open procedure always entails the possibility of infection at the exposed operating site. In addition, it would place less strain on the patient if such an operation were performed using minimally invasive techniques, that is to say by arthroscopy.

It is an object of the present invention is to make available an instrument for autologous chondrocyte transplantation with which an implantable nonwoven can be implanted in particular by arthroscopy.

### SUMMARY OF THE INVENTION

According to the invention, the object is achieved by an instrument for autologous chondrocyte transplantation, with a stamp which is used for introducing an implantable nonwoven into a cartilage and which has, on a distal end face, at least one opening to which an underpressure can be applied so that an implantable nonwoven can be held on the end face.

By means of the underpressure, the implantable nonwoven adheres to the distal end face of the stamp. The implantable nonwoven can then be suitably inserted by means of the stamp into the prepared cartilage defect. After insertion of the implantable nonwoven, the underpressure is interrupted, the stamp is lifted from the implantable nonwoven, and the latter remains in the prepared cartilage defect.

It is thus possible, with this kind of stamp-like instrument, to accurately insert an implantable nonwoven for autologous chondrocyte transplantation into a cartilage defect by minimally invasive arthroscopy.

In one embodiment of the invention, a plurality of openings are provided on the distal end face of the stamp.

By means of this measure, the implantable nonwoven is held at more than one location of the distal end face. In this way, the implantable nonwoven is held much more firmly and adheres more uniformly on the distal end face. This avoids a peeling-off of the implantable nonwoven or the formation of folds.

In one embodiment, a multiplicity of the openings are arranged in the area of the circumference of the distal end face of the stamp.

This measure ensures that the margins of the implantable nonwoven are held securely on the distal end face of the stamp and the implantable nonwoven can be inserted particularly reliably and with an exact fit into the prepared cartilage defect.

In a further embodiment of the invention, the at least one opening can be subjected to an overpressure.

After the implantable nonwoven has been fitted into the cartilage defect and the underpressure has been interrupted, it could happen that the implantable nonwoven would remain sticking to the distal end face of the stamp on account of adhesion. This can happen, for example, if the implantable nonwoven, which is a biological material, has to be kept moist.

If an overpressure is applied via the openings to the implantable nonwoven in the direction of the cartilage defect, the detachment of the implantable nonwoven from the stamp is assisted. This means that detachment can be ensured without changing the position of the already fitted implantable nonwoven.

The overpressure is applied by providing a flow of fluid through the openings, which fluid can, for example, be sterile water, sterile saline solution or sterile compressed air.

In a further embodiment of the invention, the instrument further comprises a cartilage puncher for punching out cartilage tissue.

A cartilage puncher is a particularly simple instrument for dissecting an area of cartilage damage.

In a further embodiment of the abovementioned measure, the cartilage puncher has a tubular configuration.

By means of this measure, a cartilage puncher can be used not only via arthroscopy, for punching out the cartilage defect, but also forms a channel through which further instruments can be guided to the cartilage defect.

These other instruments can include, for example, surgical spoons or curettes, which are used for removing the punched-out tissue from the bone.

Moreover, a tubular cartilage puncher can also be used for guiding the stamp through it to the cartilage defect. It can therefore also act as a targeting device for the stamp.

In a further embodiment of the abovementioned measure, the inner cross section of the cartilage puncher corresponds approximately to the cross-sectional contour of the stamp.

The stamp can therefore be used to insert an implantable nonwoven which corresponds exactly to the punch area.

An oval cross section has proven to be a suitable cross-sectional contour.

In a further embodiment of the invention, the instrument further comprises at least one nonwoven puncher for punching out the implantable nonwoven.

An implantable nonwoven can be prepared particularly easily by means of a nonwoven puncher. The punch blade of the nonwoven puncher has approximately the same contour as that of the cartilage puncher, so that a nonwoven section matching the previously prepared defect is punched out.

In one embodiment of the aforementioned measure, the nonwoven puncher has a tubular configuration.

By means of this measure, after the punching procedure, the stamp can be guided through the nonwoven puncher toward the punched-out implantable nonwoven. This makes it much easier to take up the implantable nonwoven by means of the stamp.

In a further embodiment of the abovementioned measure, the inner cross section of the nonwoven puncher corresponds approximately to the cross-sectional contour of the stamp.

By means of this measure, the stamp can be inserted with a snug fit into the nonwoven puncher and guided toward the punched-out section of nonwoven. The stamp is positioned with precise targeting over the punched-out implantable nonwoven and is able to take the latter up.

In a further embodiment of the invention, sets of stamps, cartilage punchers and nonwoven punchers are provided which are differently configured and adapted to one another.

By means of this measure, an operating surgeon is able to select, from a plurality of instrument sets, the particular set whose shape and size corresponds to the defect that is to be treated. It has been found that, with three different sizes, it is possible to cover about 90% of the usual defect sizes.

It will be appreciated that the aforementioned features and those features still to be explained below can be used not only in the respectively cited combination, but also in other combinations or singly, without departing from the scope of the present invention.

### BRIEF DESCRIPTION OF THE DRAWINGS

The invention is described and explained in more detail below on the basis of a selected illustrative embodiment and with reference to the attached drawings, in which:

FIG. 1 shows a perspective view of a stamp of an instrument for ACT,

FIG. 2 shows a perspective view of a cartilage puncher of an instrument for ACT,

FIG. 3 shows a perspective view of a nonwoven puncher of an instrument for ACT,

FIG. 4 shows a highly schematic view of a first step of a method for ACT using an instrument according to the invention,

FIG. 5 shows a highly schematic view of a second step of the method from FIG. 4,

FIG. 6 shows a highly schematic view of a third step of the method from FIG. 4,

FIG. 7 shows a highly schematic view of a fourth step of the method from FIG. 4,

FIG. 8 shows a highly schematic view of a fifth step of the method from FIG. 4,

FIG. 9 shows a highly schematic view of a sixth step of the method from FIG. 4,

FIG. 10 shows a highly schematic view of a seventh step of the method from FIG. 4,

FIG. 11 shows a highly schematic view of an eighth step of the method from FIG. 4,

FIG. 12 shows a highly schematic view of a ninth step of the method from FIG. 4, and

FIG. 13 shows a highly schematic view of a tenth step of the method from FIG. 4.

### DETAILED DESCRIPTION OF THE INVENTION

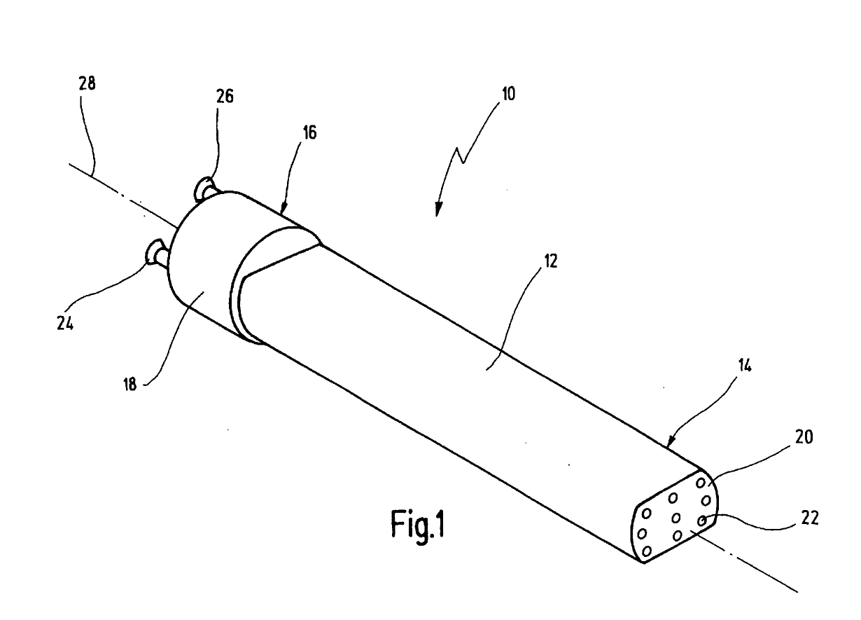

In FIG. 1, a stamp of an instrument for ACT is designated in its entirety by reference number 10.

The stamp 10 has a rod-shaped body 12 with a distal portion 14 and a proximal portion 16.

The proximal portion 16 widens to form a handle 18.

The distal portion 14 terminates in a plane end face 20 which roughly has the shape of a rectangle with rounded corners. This approximately oval shape is particularly suitable for ACT, since most cartilage defects are in the form of tears, so that in most cases an approximately oval implant has to be used.

Openings 22 are formed in the end face 20, these openings 22 being uniformly distributed and in particular being arranged in the area of the circumference. By means of these openings 22, an implantable nonwoven can be sucked onto the stamp 10. The arrangement of the openings 22 ensures that the margins of the implantable nonwoven are sucked onto the stamp 10 and adhere firmly thereto. The openings 22 are connected via bores in the body 12 to a first attachment piece 24 and to a second attachment piece 26 at the proximal portion 16 of the body 12 of the stamp 10.

The first attachment piece 24 can be connected to a source for an underpressure, for example via a hose. In this way, the openings 22 can be subjected to an underpressure, and an implantable nonwoven can be sucked onto the end face 20 of the stamp 10.

The second attachment piece 26 can be connected to a source for a sterile fluid, as a result of which a flow can be generated in the direction of the distal portion 14 of the body 12. By means of this flow, an implantable nonwoven can be detached from the end face 20 of the stamp 10. A valve (not shown here) permits switching between the different operating modes.

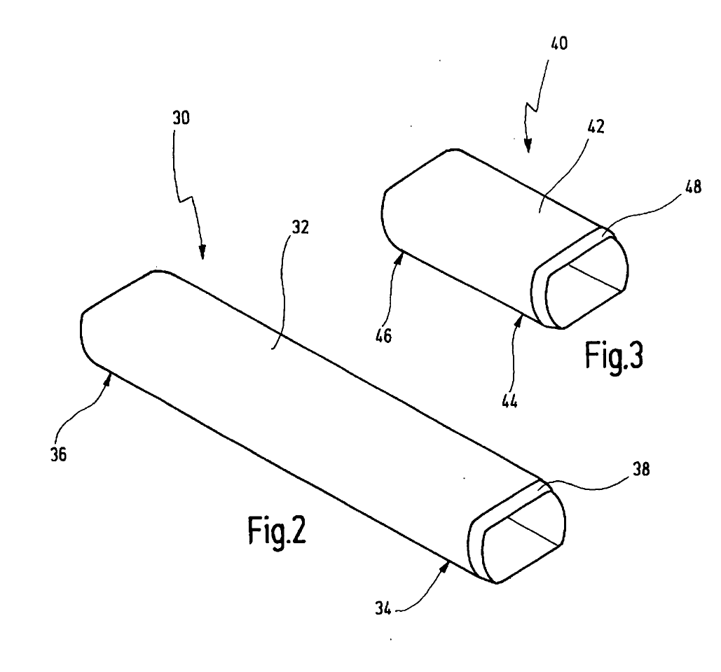

In FIG. 2, a cartilage puncher is designated in its entirety by reference number 30.

The cartilage puncher 30 has a body 32 which is of a tubular configuration. The inner cross section of the body 32 corresponds to the cross-sectional contour of the body 12 of the stamp 10 from FIG. 1.

The body 32 has a distal portion 34 and a proximal portion 36. The distal portion 34 is ground to form a punch blade 38 that extends about the whole circumference of the body 32.

In FIG. 3, a nonwoven puncher of an instrument for ACT is designated in its entirety by reference number 40.

The nonwoven puncher 40 has a tubular body 42 with a distal portion 44 and a proximal portion 46.

The body 42 essentially corresponds in structure and diameter to the body 32 of the cartilage puncher 30 from FIG. 2, the distal portion 44 likewise being ground to form a punch blade 48 extending round the whole circumference.

The punch blade 48 has the same shape and size as the punch blade 38 of the cartilage puncher 30 from FIG. 2.

This dimensioning ensures that the nonwoven puncher 40 punches out an implantable nonwoven which has the same shape and size as the cartilage area punched out by the cartilage puncher 30.

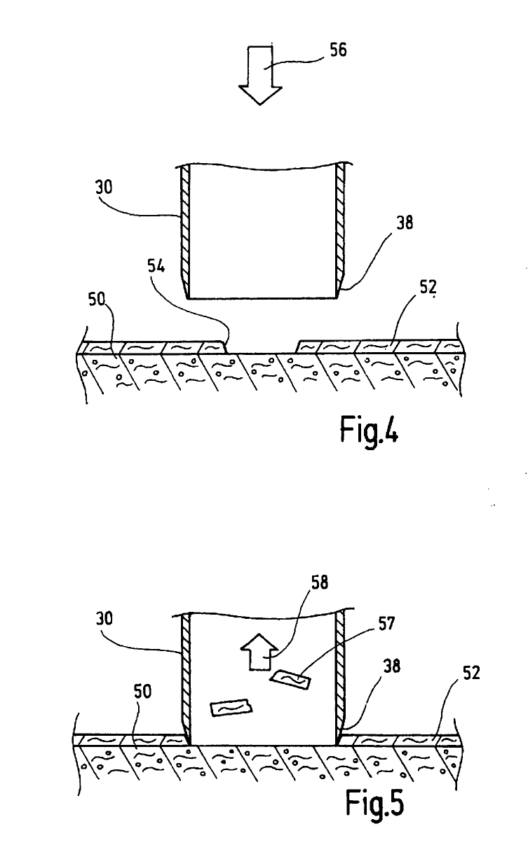

FIG. 4 shows a schematic view of a first step of a method for ACT.

Here, the cartilage puncher 30 is advanced toward a bone 50 which is covered by cartilage 52.

The bone 50 is located in the knee joint. The surface of the bone 50 is shown flat for the sake of simplicity only. The bone surfaces in the knee joint are almost exclusively curved bone surfaces.

The arthroscopic instruments used for insertion of the cartilage puncher 30, for example an arthroscope for visual monitoring, are likewise not shown, for the sake of simplicity.

The cartilage 52 has a cartilage defect 54, here in the form of a loss of cartilage tissue.

The punch blade 38 is oriented around the cartilage defect 54.

The cartilage puncher 30 is now advanced in the direction of an arrow 56 onto the bone 50 and the cartilage 52. The punch blade 38 is pressed into the cartilage 52 and cuts through it, as a result of which the margins of the cartilage defect 54 are punched out.

FIG. 5 shows that the punch blade 38 of the cartilage puncher 30 has cut through the cartilage 52 and comes to lie against the surface of the bone 50.

The pieces 57 of the cartilage 52 that have been punched out by the punch blade 38 are removed from the cartilage puncher 30 in the direction of an arrow 58 with the aid of a surgical spoon (not shown here).

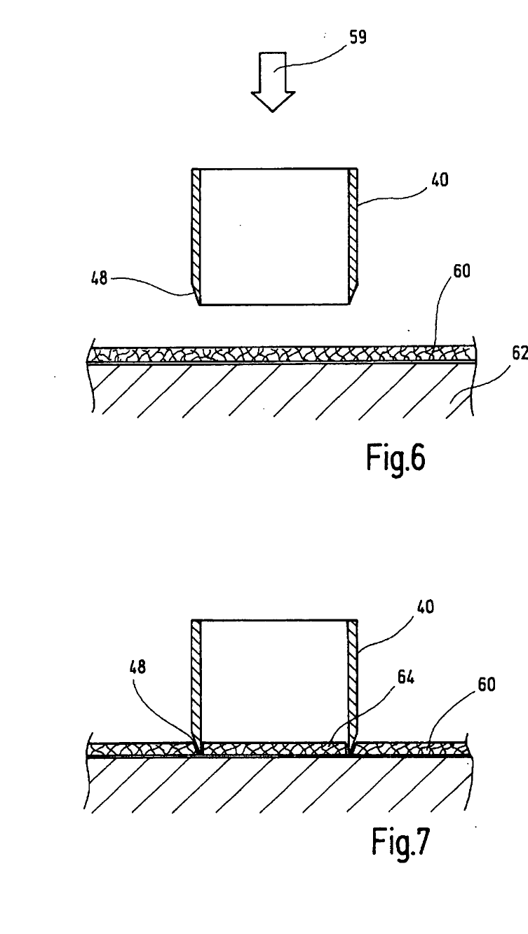

FIG. 6 shows a further step of the method for ACT. This step and the following method steps for preparation of an implantable nonwoven can be carried out before, during or after the two method steps described above.

Here, the nonwoven puncher 40 is moved in the direction of an arrow 59 toward a nonwoven 60 and is pressed into the latter.

The nonwoven 60 is a pig collagen matrix on which chondrocytes from a patient to be treated have been cultured beforehand in the laboratory. The thickness of the nonwoven 60 corresponds approximately to the thickness of the cartilage 52 that is to be treated. The nonwoven 60 rests on a support 62, here a metal plate, which is strong enough to offer resistance to the punching operation.

As will be seen from FIG. 7, the punch blade 48 has been pressed into the nonwoven 60 and has cut out an implantable nonwoven 64 from this. As a result of the dimensioning of the nonwoven puncher 40 and of the cartilage puncher 30, the implantable nonwoven 64 has the same size as the area of the cartilage 52 previously punched out by the cartilage puncher 30.

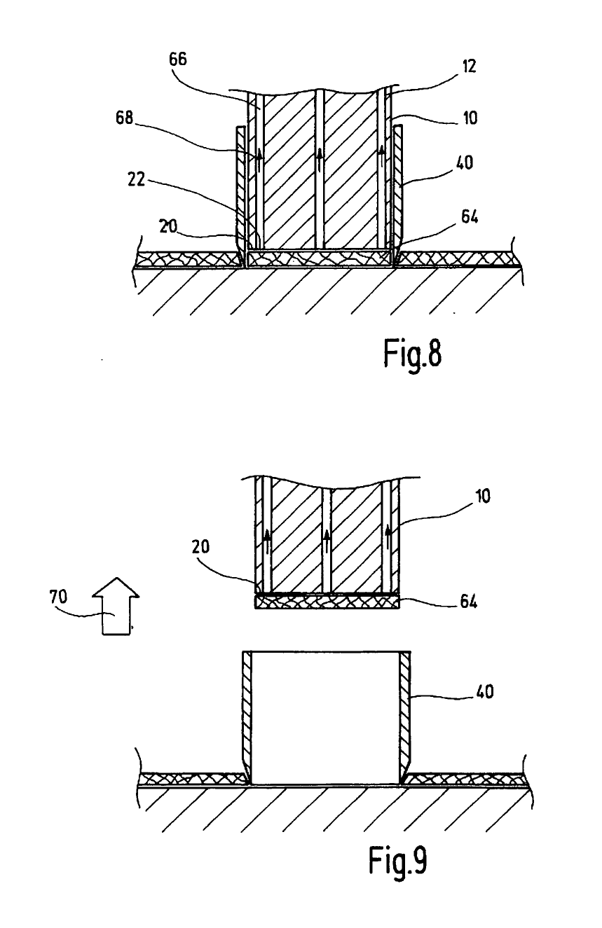

As is shown in FIG. 8, the stamp 10 is now introduced into the nonwoven puncher 40 from the proximal direction until the end face 20 of the stamp 10 comes to lie on the implantable nonwoven 64.

As a result of the dimensioning of the stamp 10 and of the nonwoven puncher 40, the stamp 10 is introduced with a snug fit into the nonwoven puncher 40. This ensures that the end face 20 comes to lie exactly above the implantable nonwoven 64.

An underpressure is now applied in the direction of the arrows 68 within bores 66 in the body 12 of the stamp 10.

The bores 66 are connected to the openings 22 in the end face 20 of the stamp 10. By means of this underpressure, the implantable nonwoven 64 that has been punched out beforehand is sucked onto the end face 20 of the stamp 10.

When the implantable nonwoven 64 is sucked onto the end face 20 of the stamp 10, the latter can, as is shown in FIG. 9, be withdrawn from the nonwoven puncher 40 in the direction of an arrow 70, that is to say in the proximal direction. In doing so, the implantable nonwoven 64 is removed from the nonwoven puncher 40 and remains securely adhering to the end face 20 of the stamp 10. This permits the further handling of the combination comprising stamp 10 and implantable nonwoven 64, the latter adhering firmly in place.

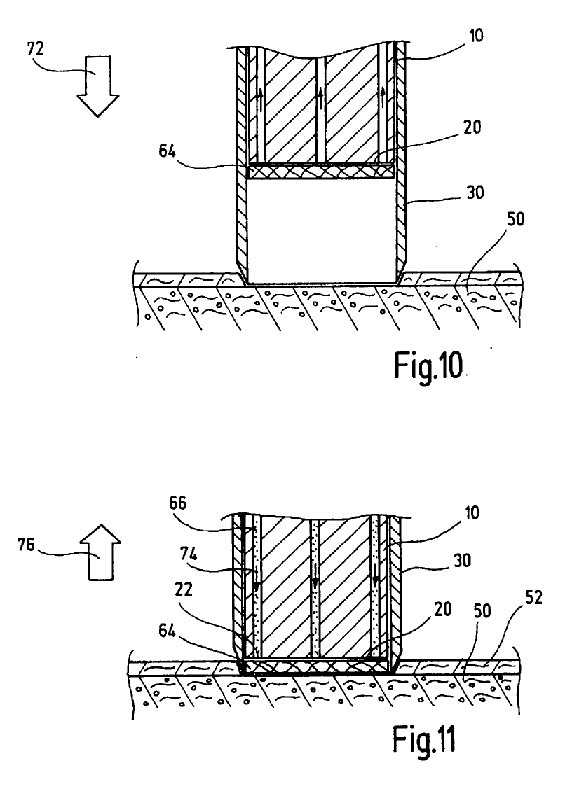

The stamp 10 and the adhering implantable nonwoven 64 are now introduced from the proximal direction into the cartilage puncher 30 which is still in place (as shown in FIG. 5) in the cartilage 50, as will be seen from FIG. 10.

The implantable nonwoven 64 is introduced, as shown in FIG. 11, into the prepared cartilage defect and bears on the surface of the bone 50. Its height means it is approximately flush with the cartilage 52. In a next step, a flow of fluid is now generated in the bores 66 in the direction of the arrows 74. This is done here by introducing sterile water. By means of this flow of fluid, an overpressure is generated at the openings 22 and helps detach the implantable nonwoven 64 from the end face 20 of the stamp 10.

The stamp 10 is then withdrawn from the cartilage puncher 30 in the direction of an arrow 76.

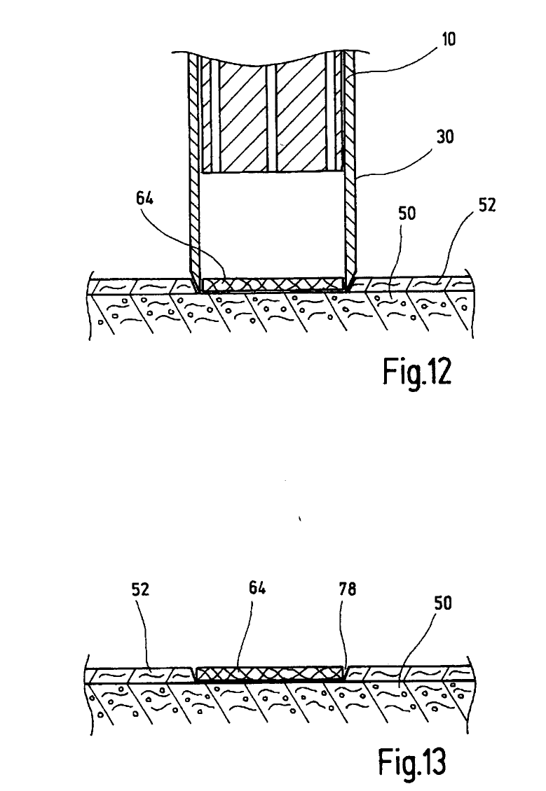

As is shown in FIG. 12, the implantable nonwoven 64 remains bearing on the surface of the bone 50, while the stamp 10 has already been largely withdrawn from the cartilage puncher 30. In a step following on from the withdrawal of the stamp, the cartilage puncher 30 is now also removed from the body of a patient.

FIG. 13 shows the situation after completion of the ACT method.

After removal of the stamp 10 and of the cartilage puncher 30, only the implantable nonwoven 64 remains in the patient's body. It now lies flush with the surface of the bone 50 and has approximately the same height as the cartilage 52. At the places where the blade 38 of the cartilage puncher 30 has penetrated into the cartilage 52, a small gap 78 still remains where the implantable nonwoven 64 can be bonded to the cartilage 52 by means of fibrin adhesive.

The cartilage cells present in the implantable nonwoven 64 grow in the course of the healing process and attach themselves to the already existing cartilage 52. The matrix of the implantable nonwoven 64, that is to say the pig collagen, is at the same time degraded. After a certain time, the implantable nonwoven 64 is then completely replaced by new cartilage tissue, and the cartilage damage is completely rectified.

To promote the attachment of the implantable nonwoven 64, an instrument as described in a parallel German patent application, file no. 10 2005 010 989.6, filed on Mar. 3, 2005, with the title “Medical instrument for performing microfractures in a bone”, can be used to perform “microfracturing” on the bone exposed in FIG. 5 which document is fully incorporated by reference as part of that description. The clot emerging from the microfractures can distribute within the implantable nonwoven and form cartilage tissue.

What is claimed is:

1. A medical instrument for autologous chondrocyte transplantation, comprising a stamp for introducing an implantable nonwoven into a cartilage, having a distal end face, at least one opening being provided in said distal end face to which opening an underpressure can be applied for holding an implantable nonwoven on said end face.

2. The instrument of claim 1, wherein a plurality of openings are provided in said distal end face of said stamp.

3. The instrument of claim 2, wherein a multiplicity of said openings are arranged in an area of a circumference of said distal end face of said stamp.

4. The instrument of claim 1, wherein said at least one opening can be subjected to an overpressure.

5. The instrument of claim 1, further comprising at least one cartilage puncher for punching out a cartilage tissue.

6. The instrument of claim 5, wherein said at least one cartilage puncher has a tubular configuration.

7. The instrument of claim 6, wherein an inner cross section of said cartilage puncher corresponds approximately to a cross-sectional contour of said stamp.

8. The instrument of claim 1, further comprising at least one nonwoven puncher for punching out an implantable nonwoven.

9. The instrument of claim 8, wherein said at least one nonwoven puncher has a tubular configuration.

10. The instrument of claim 9, wherein an inner cross section of said nonwoven puncher corresponds approximately to a cross-sectional contour of said stamp.
